(19) United States
(12) Patent Application Publication
(10) Pub. No.: US 2015/0064224 A1
(43) Pub. Date: Mar. 5, 2015

(54) Use of Certain Water-Insoluble Porous Polymeric Particles in Spherical Form as SPF Boosters

(71) Applicant: L'OREAL, Paris (FR)

(72) Inventors: Jun Hua TONG, Jersey City, NJ (US); Paula CZIRYAK, Eatontown, NJ (US)

(73) Assignee: L'OREAL, Paris (FR)

(21) Appl. No.: 14/013,888

(22) Filed: Aug. 29, 2013

(51) Int. Cl. A61K 8/02; A61Q 17/04

(52) U.S. Cl. CPC A61K 8/025; A61Q 17/04
USPC 424/401; 424/59; 424/60

(57) ABSTRACT

The present invention relates to a composition intended for protecting the skin and/or hair against ultraviolet radiation containing: (a) at least one photoprotective system capable of screening out UV radiation; and (b) water-insoluble polymeric porous particles in spherical form.

### BACKGROUND OF THE INVENTION

The present invention relates to a composition intended for protecting the skin and/or hair against ultraviolet radiation, containing, in a cosmetically acceptable aqueous medium:

(a) at least one photoprotective system capable of screening out UV radiation; and

(b) certain water-insoluble porous polymeric particles in spherical form.

The present invention also relates to the use of certain water-insoluble porous polymeric particles in a composition comprising, in a cosmetically acceptable medium, at least one photoprotective system capable of screening out UV radiation.

It is known that light radiation with wavelengths of between 280 nm and 400 nm permits tanning of the human epidermis and that rays with wavelengths of between 280 and 320 nm, which are known as UV-B rays, cause skin burns and erythema that can harm the development of a natural tan; this UV-B radiation should thus be screened out.

It is also known that UV-A rays, with wavelengths of between 320 and 400 nm, which cause tanning of the skin, are liable to induce impairment thereof, especially in the case of sensitive skin or of skin that is continually exposed to solar radiation. UV-A rays in particular cause a loss of skin elasticity and the appearance of wrinkles, leading to premature ageing. They promote the onset of the erythematous reaction or amplify this reaction in the case of certain individuals, and may even be the cause of phototoxic or photoallergic reactions. It is thus desirable also to screen out UV-A radiation.

Many photoprotective (UV-A and/or UV-B) cosmetic compositions for the skin have been proposed to date. formulations that afford for the users easy application to skin are most particularly sought.

These sunscreen compositions are quite often in the form of an emulsion of oil-in-water type (i.e. a cosmetically acceptable support constituted of an aqueous dispersing continuous phase and of an oily dispersed discontinuous phase) that contains, in varying concentrations, one or more standard lipophilic and/or hydrophilic organic screening agents capable of selectively absorbing the harmful UV radiation, these screening agents (and the amounts thereof) being selected as a function of the desired sun protection factor, the sun protection factor (SPF) being expressed mathematically as the ratio of the dose of UV radiation required to reach the erythema-forming threshold with the UV-screening agent, to the dose of UV radiation required to reach the erythema-forming threshold without UV-screening agent.

Thus, there is still a significant need to find sunscreen products that have a sufficiently high sun protection factor. High sun protection factors may be reached by incorporating more screening agents. This is not always achievable, despite the addition of more screening agents. Furthermore, such a strategy may result in impairment of the composition's comfort (tacky, coarse effect and/or greasy effect).

After considerable research conducted in the field of photoprotection mentioned above, the Applicant has now discovered, surprisingly, that the addition of certain water-insoluble porous polymeric particles, in a composition containing at least one system for screening out UV radiation, made it possible to increase its photoprotection efficacy and in particular its sun protection factor.

This discovery forms the basis of the present invention.

Thus, in accordance with a first object of the present invention, novel compositions for protecting the skin and/or hair against ultraviolet radiation are proposed which contain, in a cosmetically acceptable vehicle:

(a) at least one photoprotective system capable of screening out UV radiation; and

(b) certain water-insoluble porous polymeric particles in spherical form.

According to the invention, the term “photoprotective system capable of screening out UV radiation” is generally intended to denote any compound or any combination of compounds which, via mechanisms known per se of absorption and/or reflection and/or scattering of UV-A and/or UV-B radiation, makes it possible to prevent, or at least to limit, the contact of said radiation with a surface (skin or hair) onto which this or these compound(s) have been applied. In other words, these compounds may be UV-absorbing photoprotective organic screening agents or UV-scattering and/or UV-reflecting mineral pigments, and also mixtures thereof.

The term “cosmetically acceptable” means compatible with the skin and/or its integuments, which has a pleasant colour, odour and feel and which does not cause any unacceptable discomfort (stinging, tautness or redness) liable to put the consumer off using this composition.

Yet another object of the present invention lies in the use of certain water-insoluble porous polymeric particles in a composition comprising, in a cosmetically acceptable support, at least one photoprotective system capable of screening out UV radiation, for the purpose of increasing the composition's sun protection factor (SPF).

Other features, aspects and advantages of the present invention will emerge on reading the detailed description that follows.

Preferably, the certain water-insoluble porous polymeric particles in spherical form are those having an average particle size ranging from about 0.1 to about 100, preferably from about 1 to about 50, more preferably greater than 5 and especially from 5 to about 15, optimally from about 6 to about 10 micron.

Organic polymers or copolymers are the preferred materials and can be formed from monomers including the acid, salt or ester forms of acrylic acid, methacrylic acid, methylacrylate, ethylacrylate, ethylene, propylene, vinylidene chloride, acrylonitrile, maleic acid, vinyl pyrrolidone, styrene, butadiene and mixtures thereof. The polymers are especially useful in cross-linked form. Oil Absorbance (castor oil) is a measure of porosity and may range from about 90 to about 500, preferably from about 100 to about 200, most preferably from about 120 to about 180 ml/100 grams. Surface area of the particles most preferably is 8 m²/gram.

Particularly preferred porous polymers include polymethylmethacrylate and cross-linked polystyrene. Most preferred is polymethyl methacrylate commercially available as Covabead® LH 85 from Sensient Cosmetic Technologies or GanzPearl® GMP-0800 commercially available from Presperse, Inc. known also by their INCI name of Methyl Methacrylate Crosspolymer.

Amounts of the water-insoluble polymeric porous particles in spherical form may range from about 1 to about 30%, preferably from about 1 to about 10%, optimally from about 2 to about 4% by weight, based on the total weight of the composition.

According to the invention, the photoprotective system may be comprised of one or more hydrophilic, lipophilic or insoluble organic screening agents and/or one or more mineral pigments. Preferentially, it will be constituted of at least one hydrophilic, lipophilic or insoluble organic UV-screening agent.

The organic UV-screening agents are chosen especially from cinnamic derivatives; anthranilates; salicylic derivatives; dibenzoylmethane derivatives; camphor derivatives; benzophenone derivatives; β,β-diphenylacrylate derivatives; triazine derivatives; benzotriazole derivatives; benzalmalonate derivatives, especially those cited in patent U.S. Pat. No. 5,624,663; benzimidazole derivatives; imidazolines; bis-benzoazolyl derivatives as described in patents EP669323 and U.S. Pat. No. 2,463,264; p-aminobenzoic acid (PABA) derivatives; methylene bis(hydroxyphenylbenzotriazole) derivatives as described in applications U.S. Pat. No. 5,237,071, U.S. Pat. No. 5,166,355, GB2303549, DE19726184 and EP893119; benzoxazole derivatives as described in patent applications EP0832642, EP1027883, EP1300137 and DE10162844; screening polymers and screening silicones such as those described especially in patent application WO 93/04665; dimers derived from α-alkylstyrene such as those described in patent application DE 19855649; 4,4-diarylbutadienes such as those described in patent applications EP0967200, DE19746654, DE19755649, EP-A-1008586, EP1133980 and EP1133981, merocyanine derivatives such as those described in patent applications WO 04/006878, WO 05/058269 and WO 06/032741; and mixtures thereof.

As examples of complementary organic photoprotective agents, mention may be made of those denoted hereinbelow under their INCI name:

### Cinnamic Derivatives:

Ethylhexyl Methoxycinnamate sold in particular under the trade name “Parsol® MCX” by DSM Nutritional Products,

Isopropyl Methoxycinnamate,

Isoamyl Methoxycinnamate sold under the trade name “Neo Heliopan® E 1000” by Symrise,

DEA Methoxycinnamate,

Diisopropyl Methylcinnamate,

Glyceryl Ethylhexanoate Dimethoxycinnamate.

### Dibenzoylmethane Derivatives:

Butyl Methoxydibenzoylmethane sold especially under the trade name “Parsol® 1789” by DSM,

Isopropyl Dibenzoylmethane.

### Para-Aminobenzoic Acid Derivatives:

PABA,

Ethyl PABA,

Ethyl Dihydroxypropyl PABA,

Ethylhexyl dimethyl PABA sold in particular under the name “Escalol™ 507” by ISP,

Glyceryl PABA,

PEG-25 PABA sold under the name “Uvinul® P25” by BASF.

### Salicylic Derivatives:

Homosalate sold under the name “Eusolex® HMS” by Rona/EM Industries,

Ethylhexyl Salicylate sold under the name “Neo Heliopan® OS” by Symrise,

Dipropylene Glycol Salicylate sold under the name “Dipsal™” by Scher,

TEA Salicylate sold under the name “Neo Heliopan® TS” by Symrise.

### ββ-Diphenylacrylate Derivatives:

Octocrylene sold in particular under the trade name “Uvinul® N539” by BASF,

Etocrylene sold in particular under the trade name “Uvinul® N35” by BASF.

### Benzophenone Derivatives:

Benzophenone-1 sold under the trade name “Uvinul® 400” by BASF,

Benzophenone-2 sold under the trade name “Uvinul® D50” by BASF,

Benzophenone-3 or Oxybenzone sold under the trade name “Uvinul® M40” by BASF,

Benzophenone-4 sold under the trade name “Uvinul® MS40” by BASF,

Benzophenone-5,

Benzophenone-6 sold under the trade name “Helisorb® 11” by Norquay,

Benzophenone-8 sold under the trade name “Spectra-Sorb UV-24” by American Cyanamid,

Benzophenone-9 sold under the trade name “Uvinul® DS-49” by BASF,

Benzophenone-12,

n-Hexyl 2-(4-diethylamino-2-hydroxybenzoyl)benzoate sold under the trade name “Uvinul® A+” or as a mixture with octyl methoxycinnamate under the trade name “Uvinul® A+B” by BASF.

### Benzylidenecamphor Derivatives:

3-Benzylidene Camphor manufactured under the name “Mexoryl™ SD” by Chimex,

4-Methylbenzylidene Camphor sold under the name “Eusolex® 6300” by Merck,

Benzylidene Camphor Sulfonic Acid manufactured under the name “Mexoryl™ SL” by Chimex,

Camphor Benzalkonium Methosulfate manufactured under the name “Mexoryl™ SO” by Chimex,

Terephthalylidene Dicamphor Sulfonic Acid manufactured under the name “Mexoryl™ SX” by Chimex,

Polyacrylamidomethyl Benzylidene Camphor manufactured under the name “Mexoryl™ SW” by Chimex.

### Phenylbenzimidazole Derivatives:

Phenylbenzimidazole Sulfonic Acid sold in particular under the trade name “Eusolex® 232” by Merck,

Disodium Phenyl Dibenzimidazole Tetrasulfonate sold under the trade name “Neo Heliopan® AP” by Symrise.

### Phenylbenzotriazole Derivatives:

Drometrizole Trisiloxane sold under the name “Silatrizole” by Rhodia Chimie,

Methylene bis-Benzotriazolyl Tetramethylbutyl-phenol sold in solid form under the trade name “MIXXIM BB/100” by Fairmount Chemical, or in micronized form as an aqueous dispersion under the trade name “Tinosorb M” by Ciba Specialty Chemicals.

### Triazine Derivatives:

bis-Ethylhexyloxyphenol Methoxyphenyl Triazine sold under the trade name “Tinosorb S” by Ciba Geigy,

Ethylhexyl Triazone sold in particular under the trade name “Uvinul® T150” by BASF,

Diethylhexyl Butamido Triazone sold under the trade name “Uvasorb® HEB” by Sigma 3V,

2,4,6-tris(dineopentyl 4′-aminobenzalmalonate)-s-triazine,

2,4,6-tris(diisobutyl 4′-aminobenzalmalonate)-s-triazine,

2,4-bis(dineopentyl 4′-aminobenzalmalonate)-6-(n-butyl 4′-aminobenzoate)-s-triazine,

symmetrical triazine screening agents described in patent U.S. Pat. No. 6,225,467, patent application WO 2004/085412 (see compounds 6 and 9) or the document “Symmetrical Triazine Derivatives” IP.COM Journal, IP.COM Inc., West Henrietta, N.Y., US (20 Sep. 2004), especially 2,4,6-tris(biphenyl)-1,3,5-triazines (in particular 2,4,6-tris(biphenyl-4-yl)-1,3,5-triazine and 2,4,6-tris(terphenyl)-1,3,5-triazine, which is included in patent applications WO 06/035000, WO 06/034982, WO 06/034991, WO 06/035007, WO 2006/034992 and WO 2006/034985).

### Anthranilic Derivatives:

Menthyl Anthranilate sold under the trade name “Neo Heliopan® MA” by Symrise.

### Imidazoline Derivatives:

Ethylhexyl Dimethoxybenzylidene Dioxoimidazoline Propionate.

### Benzalmalonate Derivatives:

Polyorganosiloxane containing benzalmalonate functions, for instance Polysilicone-15, sold under the trade name “Parsol® SLX” by DSM Nutritional Products.

### 4,4-Diarylbutadiene Derivatives:

1,1-dicarboxy(2,2′-dimethylpropyl)-4,4-diphenyl-butadiene.

### Benzoxazole Derivatives:

2,4-bis[5-(1-dimethylpropyl)benzoxazol-2-yl-(4-phenyl)imino]-6-(2-ethylhexyl)imino-1,3,5-triazine sold under the name Uvasorb® K2A by Sigma 3V, and mixtures thereof.

The preferential organic screening agents are chosen from:

Ethylhexyl Methoxycinnamate,

Ethylhexyl Salicylate,

Homosalate,

Butyl Methoxydibenzoylmethane,

Octocrylene,

Phenylbenzimidazole Sulfonic Acid,

Benzophenone-3,

Benzophenone-4,

Benzophenone-5,

n-Hexyl 2-(4-diethylamino-2-hydroxybenzoyl)benzoate,

4-Methylbenzylidene Camphor,

Terephthalylidene Dicamphor Sulfonic Acid,

Disodium Phenyl Dibenzimidazole Tetrasulfonate,

Methylene Bis-Benzotriazolyl Tetramethylbutylphenol,

Bis-Ethylhexyloxyphenol Methoxyphenyl Triazine,

Ethylhexyl triazone,

Diethylhexyl Butamido Triazone,

2,4,6-Tris(dineopentyl 4′-aminobenzalmalonate)-s-triazine,

2,4,6-Tris(diisobutyl 4′-aminobenzalmalonate)-s-triazine,

2,4-Bis(dineopentyl 4′-aminobenzalmalonate)-6-(n-butyl 4′-aminobenzoate)-s-triazine,

2,4,6-Tris(biphenyl-4-yl)-1,3,5-triazine,

2,4,6-Tris(terphenyl)-1,3,5-triazine,

Drometrizole Trisiloxane,

Polysilicone-15,

1,1-dicarboxy(2,2′-dimethylpropyl)-4,4-diphenylbutadiene,

2,4-bis[5-1(dimethylpropyl)benzoxazol-2-yl-(4-phenyl)imino]-6-(2-ethylhexyl)imino-1,3,5-triazine,

and mixtures thereof.

The inorganic UV screening agents used in accordance with the present invention are metal oxide pigments. More preferentially the inorganic UV screening agents of the invention are metal oxide particles having a mean elementary particle size of less than or equal to 500 nm, more preferably of between 5 nm and 500 nm and more preferably still of between 10 nm and 100 nm, and preferably between 15 nm and 50 nm.

They may be selected especially from titanium oxides, zinc oxides, iron oxides, zirconium oxides, cerium oxides or mixtures thereof.

Such coated or uncoated metal oxide pigments are described in particular in the patent application EP-A-0 518 773. Commercial pigments that may be mentioned include the products sold by the companies Kemira, Tayca, Merck and Degussa.

The metal oxide pigments may be coated or uncoated.

The coated pigments are pigments that have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminium salts of fatty acids, metal alkoxides (of titanium or of aluminium), polyethylene, silicones, proteins (collagen, elastin), alkanolamines, silicon oxides, metal oxides or sodium hexametaphosphate.

The coated pigments are more particularly titanium oxides that have been coated:

with silica, such as the product “Sunveil” from the company Ikeda,

with silica and iron oxide, such as the product “Sunveil F” from the company Ikeda,

with silica and alumina, such as the products “Microtitanium Dioxide MT 500 SA” and “Microtitanium Dioxide MT 100 SA” from the company Tayca and “Tioveil™ ” from the company Tioxide,

with alumina, such as the products “Tipaque TTO-55 (B)” and “Tipaque TTO-55 (A)” from the company Ishihara and “UVT 14/4” from the company Kemira,

with alumina and aluminium stearate, such as the products “Microtitanium Dioxide MT 100 T, MT 100 TX, MT 100 Z and MT-01” from the company Tayca, the products “Solaveil™ CT-10 W” and “Solaveil™ CT 100”, from the company Uniqema and the product “Eusolex® T-AVO” from the company Merck,

with silica, alumina and alginic acid, such as the product “MT-100 AQ” from the company Tayca,

with alumina and aluminium laurate, such as the product “Microtitanium Dioxide MT 100 S” from the company Tayca,

with iron oxide and iron stearate, such as the product “Microtitanium Dioxide MT 100 F” from the company Tayca,

with zinc oxide and zinc stearate, such as the product “BR 351” from the company Tayca,

with silica and alumina and treated with a silicone, such as the products “Microtitanium Dioxide MT 600 SAS”, “Microtitanium Dioxide MT 500 SAS” or “Microtitanium Dioxide MT 100 SAS” from the company Tayca,

with silica, alumina and aluminium stearate and treated with a silicone, such as the product “STT-30-DS” from the company Titan Kogyo,

with silica and treated with a silicone, such as the product “UV-Titan X 195” from the company Kemira,

with alumina and treated with a silicone, such as the products “Tipaque TTO-55 (S)” from the company Ishihara or “UV Titan M 262” from the company Kemira,

with triethanolamine, such as the product “STT-65-S” from the company Titan Kogyo,

with stearic acid, such as the product “Tipaque TTO-55 (C)” from the company Ishihara,

with sodium hexametaphosphate, such as the product “Microtitanium Dioxide MT 150 W” from the company Tayca.

TiO₂ treated with octyltrimethylsilane sold under the trade name “T 805” by the company Degussa Silices,

TiO₂ treated with a polydimethylsiloxane sold under the trade name “70250 Cardre UF TiO2SI3” by the company Cardre,

anatase/rutile TiO₂ treated with a polydimethylhydrogenosiloxane sold under the trade name “Microtitanium Dioxide USP Grade Hydrophobic” by the company Color Techniques.

The uncoated titanium oxide pigments are sold, for example, by the company Tayca under the trade names “Microtitanium Dioxide MT 500 B” or “Microtitanium Dioxide MT 600 B”, by the company Degussa under the name “P 25”, by the company Wackher under the name “Transparent titanium oxide PW”, by the company Miyoshi Kasei under the name “UFTR”, by the company Tomen under the name “ITS” and by the company Tioxide under the name “Tioveil™ AQ”.

The uncoated zinc oxide pigments are, for example:

those sold under the name “Z-Cote®” by the company Sunsmart;

those sold under the name “Nanox®” by the company Elementis;

those sold under the name “Nanogard™ WCD 2025” by the company Nanophase Technologies.

The coated zinc oxide pigments are, for example:

those sold under the name “Zinc Oxide CS-5” by the company Toshibi (ZnO coated with polymethylhydrogensiloxane);

those sold under the name “Nanogard™ Zinc Oxide FN” by the company Nanophase Technologies (as a 40% dispersion in Finsolv® TN, C₁₂-C₁₅ alkyl benzoate);

those sold under the name “Daitopersion Zn-30” and “Daitopersion Zn-50” by the company Daito (dispersions in cyclopolymethylsiloxane/oxyethylenated polydimethylsiloxane, containing 30% or 50% of nanozinc oxides coated with silica and polymethylhydrogensiloxane);

those sold under the name “NFD Ultrafine ZnO” by the company Daikin (ZnO coated with perfluoroalkyl phosphate and copolymer based on perfluoroalkylethyl as a dispersion in cyclopentasiloxane);

those sold under the name “SPD-Z1” by the company Shin-Etsu (ZnO coated with silicone-grafted acrylic polymer, dispersed in cyclodimethylsiloxane);

those sold under the name “Escalol™ Z100” by the company ISP (alumina-treated ZnO dispersed in an ethylhexyl methoxycinnamate/PVP-hexadecene copolymer/methicone mixture);

those sold under the name “Fuji ZnO-SMS-10” by the company Fuji Pigment (ZnO coated with silica and polymethylsilsesquioxane);

those sold under the name “Nanox® Gel TN” by the company Elementis (ZnO dispersed at a concentration of 55% in C₁₂-C₁₅ alkyl benzoate with hydroxystearic acid polycondensate).

The uncoated cerium oxide pigments may be for example those sold under the name “Colloidal Cerium Oxide” by the company Rhone-Poulenc.

The uncoated iron oxide pigments are sold, for example, by the company Arnaud under the names “Nanogard™ WCD 2002 (FE 45B)”, “Nanogard™ Iron FE 45 BL AQ”, “Nanogard™ FE 45R AQ” and “Nanogard™ WCD 2006 (FE 45R)” or by the company Mitsubishi under the name “TY-220”.

The coated iron oxide pigments are sold, for example, by the company Arnaud under the names “Nanogard™ WCD 2008 (FE 45B FN)”, “Nanogard™ WCD 2009 (FE 45B 556)”, “Nanogard™ FE 45 BL 345” and “Nanogard™ FE 45 BL” or by the company BASF under the name “Transparent Iron Oxide”.

Mention may also be made of mixtures of metal oxides, especially of titanium dioxide and of cerium dioxide, including the silica-coated equal-weight mixture of titanium dioxide and of cerium dioxide, sold by the company Ikeda under the name “Sunveil A”, and also the alumina-, silica- and silicone-coated mixture of titanium dioxide and of zinc dioxide, such as the product “M 261” sold by the company Kemira, or the alumina-, silica- and glycerol-coated mixture of titanium dioxide and of zinc dioxide, such as the product “M 211” sold by the company Kemira.

According to the invention, coated or uncoated titanium oxide pigments are particularly preferred.

The photoprotective system according to the invention is preferably present in the compositions according to the invention in a content ranging from 0.1% to 40% by weight and in particular from 5% to 25% by weight relative to the total weight of the composition.

The aqueous compositions in accordance with the present invention may also comprise standard cosmetic adjuvants chosen especially from fatty substances, organic solvents, ionic or nonionic, hydrophilic or lipophilic thickeners, demulcents, humectants, opacifiers, stabilizers, cosmetic or dermatological active agents, emollients, silicones, antifoaming agents, preservatives, anionic, cationic, nonionic, zwitterionic or amphoteric surfactants, fillers, polymers, propellants, acidifying or basifying agents or any other ingredient usually used in cosmetics and/or dermatology.

The fatty substances may be constituted of an oil or a wax other than the apolar waxes as defined above, or mixtures thereof. The term “oil” means a compound that is liquid at room temperature. The term “wax” means a compound that is solid or substantially solid at room temperature and whose melting point is generally greater than 35° C.

Oils that may be mentioned include mineral oils (paraffin); plant oils (sweet almond oil, macadamia oil, blackcurrant seed oil or jojoba oil); synthetic oils such as perhydrosqualene, alcohols, fatty acids or fatty esters such as the C₁₂-C₁₅ alkyl benzoate sold under the trade name “Finsolv® TN” or “Witconol TN” by the company Witco, 2-ethylphenyl benzoate such as the commercial product sold under the name X-Tend 226® by the company ISP, octyl palmitate, isopropyl lanolate and triglycerides, including capric/caprylic acid triglycerides, and dicaprylyl carbonate sold under the name “Cetiol® CC” by the company Cognis, oxyethylenated or oxypropylenated fatty esters and ethers; silicone oils (cyclomethicone and polydimethylsiloxanes, or PDMS) or fluoro oils, polyalkylenes and trialkyl trimellitates such as tridecyl trimellitate.

Waxy compounds that may be mentioned include carnauba wax, beeswax, hydrogenated castor oil, polyethylene waxes and polymethylene waxes, for instance the product sold under the name Cirebelle 303 by the company Sasol.

Among the organic solvents that may be mentioned are lower alcohols and polyols. The latter may be chosen from glycols and glycol ethers, for instance ethylene glycol, propylene glycol, butylene glycol, dipropylene glycol or diethylene glycol.

Hydrophilic thickeners that may be mentioned include carboxyvinyl polymers such as the Carbopol® products (carbomers) and the Pemulen™ products (acrylate/C₁₀-C₃₀-alkylacrylate copolymer); polyacrylamides, for instance the crosslinked copolymers sold under the names Sepigel™ 305 (CTFA name: polyacrylamide/C13-14 isoparaffin/Laureth 7) or Simulgel™ 600 (CTFA name: acrylamide/sodium acryloyldimethyltaurate copolymer/isohexadecane/polysorbate 80) by the company SEPPIC; 2-acrylamido-2-methylpropanesulphonic acid polymers and copolymers, which are optionally crosslinked and/or neutralized, for instance the poly(2-acrylamido-2-methylpropanesulphonic acid) sold by the company Clariant under the trade name “Hostacerin® AMPS” (CTFA name: ammonium polyacryloyldimethyltaurate) or Simulgel™ 800 sold by the company SEPPIC (CTFA name: sodium polyacryloyldimethyltaurate/polysorbate 80/sorbitan oleate); copolymers of 2-acrylamido-2-methylpropanesulphonic acid and of hydroxyethyl acrylate, for instance Simulgel™ NS and Sepinov™ EMT 10 sold by the company SEPPIC; cellulose-based derivatives such as hydroxyethyl cellulose; polysaccharides and especially gums such as xanthan gum; water-soluble or water-dispersible silicone-based derivatives such as acrylic silicones, silicone polyethers and cationic silicones, and mixtures thereof.

Lipophilic thickeners that may be mentioned include synthetic polymers such as poly(C₁₀-C₃₀ alkyl acrylates) sold under the name “Intelimer® IPA 13-1” and “Intelimer® IPA 13-6” by the company Landec, or modified clays such as hectorite and its derivatives, for instance the products sold under the Bentone names.

As will be appreciated, a person skilled in the art will take care to select the optional additional compound(s) mentioned above and/or the amounts thereof such that the advantageous properties intrinsically associated with the compositions in accordance with the invention are not, or are not substantially, adversely affected by the envisaged addition(s).

The compositions according to the invention may be prepared according to techniques that are well known to those skilled in the art. They may be in particular in the form of a simple or complex emulsion (O/W, W/O, O/W/O or W/O/W) such as a cream or a milk; or in the form of a lotion. They may optionally be packaged as an aerosol and may be in the form of a mousse or a spray.

The compositions according to the invention are preferably in the form of an oil-in-water or water-in-oil emulsion.

The emulsions generally contain at least one emulsifier chosen from amphoteric, anionic, cationic or nonionic emulsifiers, which are used alone or as a mixture. The emulsifiers are appropriately chosen according to the emulsion to be obtained (W/O or O/W). The emulsions may also contain stabilizers of other types, for instance fillers, gelling polymers or thickeners.

As emulsifying surfactants that may be used for the preparation of the W/O emulsions, examples that may be mentioned include sorbitan, glycerol or sugar alkyl esters or ethers; silicone surfactants, for instance dimethicone copolyols, such as the mixture of cyclomethicone and of dimethicone copolyol, sold under the name “DC® 5225 C” by the company Dow Corning, and alkyldimethicone copolyols such as laurylmethicone copolyol sold under the name “Dow Corning® 5200 Formulation Aid” by the company Dow Corning; cetyldimethicone copolyol, such as the product sold under the name Abil® EM 90R by the company Goldschmidt, and the mixture of cetyldimethicone copolyol, of polyglyceryl isostearate (4 mol) and of hexyl laurate, sold under the name Abil® WE 09 by the company Goldschmidt. One or more co-emulsifiers may also be added thereto, which may be chosen advantageously from the group comprising polyol alkyl esters. Mention may also be made of Octyldodecanol (and) Octyldodecyl Xyloside (Fluidanov 20X), Polyglyceryl-2 Dipolyhydroxystearate (Dehymuls® PGPH), Polyglyceryl-3 Ricinoleate (Akoline PGPR) and Polyglyceryl-3 Diisostearate (Lameform® TG1).

Polyol alkyl esters that may especially be mentioned include polyethylene glycol esters, for instance PEG-30 Dipolyhydroxystearate, such as the product sold under the name Arlacel™ P135 by the company ICI.

Glycerol and/or sorbitan esters that may be mentioned include, for example, polyglyceryl isostearate, such as the product sold under the name Isolan® GI 34 by the company Goldschmidt, sorbitan isostearate, such as the product sold under the name Arlacel™ 987 by the company ICI, sorbitan glyceryl isostearate, such as the product sold under the name Arlacel™ 986 by the company ICI, and mixtures thereof.

For the O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters, for instance the mixture PEG-100 Stearate/Glyceryl Stearate sold, for example, by the company ICI under the name Arlacel™ 165; oxyalkylenated (oxyethylenated and/or oxypropylenated) fatty alcohol ethers; sugar esters, for instance sucrose stearate; fatty alcohol ethers of sugars, especially alkyl polyglucosides (APGs) such as decyl glucoside and lauryl glucoside sold, for example, by the company Henkel under the respective names Plantaren® 2000 and Plantaren® 1200, cetostearyl glucoside optionally as a mixture with cetostearyl alcohol, sold, for example, under the name Montanov™ 68 by the company SEPPIC, under the name Tego® care CG90 by the company Goldschmidt and under the name Emulgade® KE3302 by the company Henkel, and also arachidyl glucoside, for example in the form of a mixture of arachidyl alcohol, behenyl alcohol and arachidyl glucoside, sold under the name Montanov™ 202 by the company SEPPIC. According to one particular embodiment of the invention, the mixture of the alkyl polyglucoside as defined above with the corresponding fatty alcohol may be in the form of a self-emulsifying composition as described, for example, in document WO-A-92/06778. Mention may also be made of lecithins and derivatives (e.g. Biophilic), sugar esters and sodium stearoyl lactylate.

Among the other emulsion stabilizers, use will more particularly be made of isophthalic acid or sulphoisophthalic acid polymers, and in particular phthalate/sulphoisophthalate/glycol copolymers, for example the diethylene glycol/phthalate/isophthalate/1,4-cyclohexane-dimethanol copolymer (INCI name: Polyester-5) sold under the name “Eastman AQ™ Polymer” (AQ35S, AQ38S, AQ55S and AQ48 Ultra) by the company Eastman Chemical.

When it is an emulsion, the aqueous phase of this emulsion may comprise a nonionic vesicular dispersion prepared according to known processes (Bangham, Standish and Watkins, J. Mol. Biol. 13, 238 (1965), FR 2 315 991 and FR 2 416 008).

The compositions according to the invention find their application in a large number of treatments, especially cosmetic treatments, of the skin, the lips and the hair, including the scalp, especially for protecting and/or caring for the skin, the lips and/or the hair, and/or for making up the skin and/or the lips.

Another subject of the present invention is constituted of the use of the compositions according to the invention as defined above for the manufacture of cosmetic products for treating the skin, the lips, the nails, the hair, the eyelashes, the eyebrows and/or the scalp, especially care products, sun protection products and makeup products.

The cosmetic compositions according to the invention may be used, for example, as makeup products.

The cosmetic compositions according to the invention may be used, for example, as a care product and/or sun protection product for the face and/or body having a liquid to semi-liquid consistency, such as milks, more or less rich creams, cream gels or pastes. They may optionally be packaged as an aerosol and may be in the form of a mousse or a spray.

The compositions according to the invention in the form of vaporizable lotions in accordance with the invention are applied to the skin or the hair in the form of fine particles by means of pressurization devices. The devices in accordance with the invention are well known to those skilled in the art and comprise non-aerosol pumps or “atomizers”, the aerosol containers comprising a propellant, and also aerosol pumps using compressed air as propellant. These pumps are described in patents U.S. Pat. No. 4,077,441 and U.S. Pat. No. 4,850,517 (which form an integral part of the content of the description).

The compositions packaged in aerosol form in accordance with the invention generally contain conventional propellants, for instance hydrofluoro compounds, dichloro-difluoromethane, difluoroethane, dimethyl ether, isobutane, n-butane, propane or trichlorofluoromethane. They are preferably present in amounts ranging from 15% to 50% by weight relative to the total weight of the composition.

A person skilled in the art will select said active agent or agents as a function of the desired effect on the skin, hair, eyelashes, eyebrows or nails.

It will be possible for the composition further to comprise at least one ingredient such as soft-focus effect fillers or agents which promote the natural colouring of the skin, for the purpose of complementing the biological effect of these active agents or providing an immediate visual anti-ageing effect.

The following examples serve to illustrate the invention without however exhibiting a limiting character. In these examples the amounts of the composition ingredients are given as weight percentages relative to the total weight of the composition.

### INVENTIVE EXAMPLES

Sunscreen formulations containing the following ingredients were prepared.

| | | | |
|---|---|---|---|
| Ingredients | Ex. 1 | Ex. 2 | Ex. 3 |

Phase A

| | | | |
|---|---|---|---|
| DEMINERALIZED WATER | QS 100 | QS 100 | QS 100 |
| CHELATING AGENT | 0.1 | 0.1 | 0.1 |
| PH ADJUSTER | 0.5 | 0.5 | 0.5 |
| PRESERVATIVES | 1.5 | 1.5 | 1.5 |

Phase B₁

| | | | |
|---|---|---|---|
| UV FILTERS | 26.79 | 26.79 | 26.79 |
| EMOLLIENTS | 14.25 | 14.25 | 14.25 |
| SURFACTANTS | 2.5 | 2.5 | 2.5 |
| OIL THICKENER | 0.2 | 0.2 | 0.2 |

Phase B₂

| | | | |
|---|---|---|---|
| SUSPENDING AGENT | 1.0 | 1.0 | 1.0 |

Phase B₃

| | | | |
|---|---|---|---|
| SILICONE | 4.92 | 4.92 | 4.92 |

Phase C

| | | | |
|---|---|---|---|
| COOLING AGENT | 2.0 | 2.0 | 2.0 |

Phase D

| | | | |
|---|---|---|---|
| AESTHETIC MODIFIERS | 8.5 | 8.5 | 8.5 |
| METHYL METHACRYLATE | 2.25 | 0.00 | 10.00 |
| CROSSPOLYMER COVABEAD ® | | | |
| LH 85 - SENSIENT) | | | |

Phase E

| | | | |
|---|---|---|---|
| ANTI-OXIDANTS | 0.3 | 0.3 | 0.3 |

Phase F

| | | | |
|---|---|---|---|
| FRAGRANCE | 0.5 | 0.5 | 0.5 |

The aqueous phase A is prepared in a beaker, which is heated to 65-75° C. and mixed until homogeneous. Then cool phase A to 45° C. The oil phase B₁ is prepared in the main beaker, which is heated to 75° C. and mixed until homogeneous. Once homogeneous, add phase B₂ and mix for 5-10 minutes, until the phase is homogeneous. Begin cooling main beaker towards room temperature. At 50° C., add phase B₃ and mix until homogeneous. At 45° C., add phase C and phase D and mix hard for 10 minutes until homogeneous. Slowly add phase A into main beaker and mix for 15-20 minutes until homogeneous. Cool entire mixture to room temperature, then add phase E and phase F and mix until homogeneous.

### COMPARATIVE PRIOR ART EXAMPLES

| | | |
|---|---|---|
| | Comparative | Comparative |
| | Prior Art | Prior Art |
| Ingredients | Ex. 1 | Ex. 2 |

Phase A

| | | |
|---|---|---|
| DEMINERALIZED WATER | QS for 100 | QS for 100 |
| DISODIUM EDTA (EBETA BD - | 0.1 | 0.1 |
| BASF) | | |
| PANTHENOL (D-PANTHENOL - BASF) | 0.2 | 0.2 |
| SODIUM LACTATE (SODIUM LACTATE | 0.1 | 0.1 |
| 60% SOLUTION - DR. PAUL | | |
| LOHMAN) | | |
| SODIUM PCA (AJIDEW NL 50 - | 0.1 | 0.2 |
| AJINOMOTO) | | |
| VITIS VINIFERA (GRAPE) FRUIT | 0.1 | 0.1 |
| EXTRACT (GRAPE ORGANIC | | |
| BIOGREEN - GREENTECH) | | |
| GLYCERIN (GLYCERINE - OLEON) | 5.0 | 5.0 |

Phase B

| | | |
|---|---|---|
| CARBOMER (CARBOPOL ® ULTREZ 10 | 0.5 | 0.5 |
| POLYMER-LUBRIZOL) | | |

Phase C

| | | |
|---|---|---|
| TRIETHANOLAMINE | 2.1 | 2.1 |
| (TRIETHANOLAMINE 99% - DOW | | |
| CHEMICAL) | | |
| DEMINERALIZED WATER | 10 | 10 |

Phase D

| | | |
|---|---|---|
| MYRISTYL ALCOHOL (LANETTE 14 - | 0.5 | 0.5 |
| COGNIS) | | |
| SORBITAN STEARATE (SPAN 60-PW - | 1.10 | 1.10 |
| CRODA) | | |
| CETYL ALCOHOL (LANETTE 16 - | 0.5 | 0.5 |
| COGNIS) | | |
| STEARYL ALCOHOL (LANETTE 18 - | 0.5 | 0.5 |
| COGNIS) | | |
| SUCROSE STEARATE (TEGOSOFT PSE | 0.5 | 0.5 |
| 141 G - EVONIK GOLDSCHMIDT) | | |
| PEG-100 STEARATE (MYRJ S100-PA - | 0.5 | 0.5 |
| CRODA) | | |
| STEARIC ACID (RADIACID 0461 - | 0.25 | 0.25 |
| OLEON) | | |
| TOCOPHERYL ACETATE (VITAMIN E | 0.2 | 0.2 |
| ACETATE CARE - BASF) | | |

Phase E

| | | |
|---|---|---|
| ETHYLHEXYL METHOXYCINNAMATE | 5.5 | 5.5 |
| (PARSOL ® MCX - DSM NUTRITIONAL | | |
| PRODUCTS) | | |
| ETHYLHEXYL SALICYLATE (NEO | 3 | 3 |
| HELIOPAN ® OS - SYMRISE) | | |
| OCTOCRYLENE (UVINUL ® N539 - | 2 | 2 |
| BASF) | | |
| BUTYL METHOXY | 2 | 2 |
| DIBENZOYLMETHANE | | |
| (PARSOL ® 1789 - DSM) | | |
| METHYL METHACRYLATE | 2 | 0 |
| CROSSPOLYMER (COVABEAD ® | | |
| LH 85 - SENSIENT) | | |

Phase F

| | | |
|---|---|---|
| ISODODECANE (ISODODECANE - | 4.5 | 4.5 |
| INEOS) | | |

Phase G

| | | |
|---|---|---|
| IODOPROPYNYL BUTYL CARBAMATE | 0.1 | 0.1 |
| (GLYCACIL L - LONZA) | | |
| PHENOXYETHANOL (SEPICIDE LD - | 0.6 | 0.6 |
| SEPPIC) | | |

Phase H

| | | |
|---|---|---|
| BISABOLOL (BISABOLOL RAC - | 0.2 | 0.2 |
| BASF) | | |
| RETINYL PALMITATE (VITAMIN A | 0.01 | 0.01 |
| PALMITATE - DSM) | | |

The base formula was prepared by charging a main beaker with Phase A. This was mixed and stirred until homogeneous at 75-80° C. Phase B was added to the stirred Phase A and mixed until dispersed. Temperature was maintained while charging Phase C into the main beaker. Agitation was continued until all components were uniformly dispersed. Phase D was then melted and mixed at 75° C. Phase E was pre-mixed and the polymethylmethacrylate particles COVABEAD® were heated at 75° C. and then added to Phase D. The resultant combined Phases D/E were then added to the main beaker under homogenization conditions for five minutes. Phase F was then added to the main batch and homogenization continued for an additional five minutes. The batch was cooled to 40-45° C. whereupon Phase G was added. Temperature was reduced to 35° C. and Phase H combined into the mixture until completely uniform.

For each of the inventive examples 1-3 and comparative examples, the sun protection factor (SPF) associated therewith was then determined. This was determined by using the in vitro method described by V. Wandel et al. in SÖFW Journal 127 (2001); this method consists in determining the monochromatic protection factors over a wavelength range from 290 to 400 nm and in calculating therefrom the sun protection factor according to a given mathematical equation. The measurement was performed with a 1 nm interval on a UV-1000S machine from the company Labsphere, 0.6 mg/cm² of product being spread onto a frosted PMMA plate. The results (mean value corresponding to 5 plates per product, 10 points per plate) are collated in Table (I) below:

TABLE (I)

| | | | | Comp | Comp |
|---|---|---|---|---|---|
| Composition | EX 1 | EX 2 | EX 3 | EX 1 | EX 2 |
| Average SPF | 77.99 | 46.15 | 100.93 | 16.67 | 11.26 |
| Coefficient of | 13.9 | 13.3 | 14.8 | 12.2 | 8.0 |
| variation (%) | | | | | |

1. A composition comprising:
(a) at least one photoprotective system capable of screening out UV radiation; and
(b) water-insoluble polymeric porous particles in spherical form;
wherein the composition is used to protect a substrate onto which its applied against ultraviolet radiation.

2. The composition of claim 1, wherein the water-insoluble polymeric porous particles in spherical form have an average particle size of from about 0.1 to about 100 microns and an oil absorbance of from about 90 to about 500 ml/100 grams.

3. The composition of claim 1, wherein the water-insoluble polymeric porous particles in spherical form have an average particle size of from about 1 to about 50 microns and an oil absorbance of from about 100 to about 200 ml/100 grams.

4. The composition of claim 1, wherein the water-insoluble polymeric porous particles in spherical form have an average particle size of from about 5 to about 15 microns and an oil absorbance of from about 120 to about 180 ml/100 grams.

5. The composition of claim 1, wherein the water-insoluble polymeric porous particles in spherical form have surface area of about 8 m²/gram.

6. The composition of claim 1, wherein the water-insoluble polymeric porous particles in spherical form are present in the composition in an amount of from about 1 to about 30% by weight, based on the total weight of the composition.

7. The composition of claim 1, wherein the water-insoluble polymeric porous particles in spherical form are present in the composition in an amount of from about 1 to about 10% by weight, based on the total weight of the composition.

8. The composition of claim 1, wherein the water-insoluble polymeric porous particles in spherical form are present in the composition in an amount of from about 2 to about 4% by weight, based on the total weight of the composition.

9. The composition of claim 1, wherein the photoprotective system is comprised of one or more hydrophilic, lipophilic or insoluble organic UV screening agents and/or one or more inorganic UV screening agents.

10. The composition of claim 9, wherein the organic UV screening agents are chosen from cinnamic derivatives; anthranilates; salicylic derivatives; dibenzoylmethane derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis-benzoazolyl derivatives; p-aminobenzoic acid (PABA) derivatives; methylene bis(hydroxyphenylbenzotriazole) derivatives; benzoxazole derivatives; screening polymers and screening silicones; dimers derived from α-alkylstyrene; 4,4-diarylbutadienes; merocyanine derivatives; and mixtures thereof.

11. The composition of claim 10, where the organic UV screening agents are chosen from:
Ethylhexyl Methoxycinnamate,
Ethylhexyl Salicylate,
Homosalate,
Butyl Methoxydibenzoylmethane,
Octocrylene,
Phenylbenzimidazole Sulfonic Acid,
Benzophenone-3,
Benzophenone-4,
Benzophenone-5,
n-Hexyl 2-(4-diethylamino-2-hydroxybenzoyl)benzoate,
4-Methylbenzylidene Camphor,
Terephthalylidene Dicamphor Sulfonic Acid,
Disodium Phenyl Dibenzimidazole Tetrasulfonate,
Methylene Bis-Benzotriazolyl Tetramethylbutylphenol,
Bis-Ethylhexyloxyphenol Methoxyphenyl Triazine,
Ethylhexyl triazone,
Diethylhexyl Butamido Triazone,
2,4,6-Tris(dineopentyl 4′-aminobenzalmalonate)-s-triazine,
2,4,6-Tris(diisobutyl 4′-aminobenzalmalonate)-s-triazine,
2,4-Bis(dineopentyl 4′-aminobenzalmalonate)-6-(n-butyl 4′-aminobenzoate)-s-triazine,
2,4,6-Tris(biphenyl-4-yl)-1,3,5-triazine,
2,4,6-Tris(terphenyl)-1,3,5-triazine,
Drometrizole Trisiloxane,
Polysilicone-15,
1,1-Dicarboxy(2,2′-dimethylpropyl)-4,4-diphenylbutadiene,
2,4-Bis[5-1(dimethylpropyl)benzoxazol-2-yl-(4-phenyl)imino]-6-(2-ethylhexyl)imino-1,3,5-triazine, and mixtures thereof.

12. The composition of claim 9, where the inorganic UV screening agents are metal oxide pigments which may be coated.

13. The composition of claim 12, where the metal oxide particles have a mean elementary particle size of less than or equal to 500 nm.

14. The composition of claim 12, where the metal oxide particles are chosen from titanium oxides, zinc oxides, iron oxides, zirconium oxides, cerium oxides or mixtures thereof.

15. A composition comprising:
(a) at least one photoprotective system capable of screening out UV radiation; and
(b) from about 2 to about 4% by weight, based on the total weight of the composition of water-insoluble polymeric porous particles in spherical form having an average particle size of from about 6 to about 10 microns, an oil absorbance of from about 120 to about 180 ml/100 grams, and a surface area of about 8 m²/gram;
wherein the composition is used to protect a substrate onto which its applied against ultraviolet radiation.

16. A method of protecting a substrate from UV radiation comprising applying a sunscreen composition onto the surface of the substrate, the sunscreen composition containing:
(a) at least one photoprotective system capable of screening out UV radiation; and
(b) from about 2 to about 4% by weight, based on the total weight of the composition of water-insoluble polymeric porous particles in spherical form having an average particle size of from about 6 to about 10 microns, an oil absorbance of from about 120 to about 180 ml/100 grams, and a surface area of about 8 m²/gram;
wherein the composition is used to protect a substrate onto which it is applied from ultraviolet radiation.

17. A method of boosting a sunscreen composition's SPF by employing therein a boosting-effective amount of water-insoluble polymeric porous particles in spherical form having an average particle size of from about 5 to about 15 microns and an oil absorbance of from about 120 to about 180 ml/100 grams.

18. The method of claim 17 wherein the boosting-effective amount of particles comprises from about 1 to about 30% by weight, based on the total weight of the composition.
